(19) United States
(12) Patent Application Publication
(10) Pub. No.: US 2022/0044777 A1
(43) Pub. Date: Feb. 10, 2022

(54) SYSTEMS AND METHODS TO AVOID UNTRACKED FOLLOW-UP RECOMMENDATIONS FOR PATIENT TREATMENT

(71) Applicant: HVR MSO LLC, Poughkeepsie, NY (US)

(72) Inventors: Richard J. Friedland, Poughkeepsie, NY (US); Wayne Wadsworth, Gardiner, NY (US); Laurie Wadsworth, Gardiner, NY (US)

(21) Appl. No.: 17/509,704

(22) Filed: Oct. 25, 2021

Related U.S. Application Data
(63) Continuation of application No. 16/296,181, filed on Mar. 7, 2019, now abandoned.
(60) Provisional application No. 62/639,996, filed on Mar. 7, 2018.

(51) Int. Cl. G16H 10/60; G16H 30/20; G16H 15/00

(52) U.S. Cl. CPC G16H 10/60; G16H 15/00; G16H 30/20

(57) ABSTRACT

A system and method to capture, track, and ensure recommended follow up care for patient treatment is rendered is disclosed, comprising feeding all radiology reports into a secure database through an integration engine. The radiology reports are read in the database and sorted as requested by the user. This sorting includes sorting by the inclusion of an actionable finding, if any, and by a recommendation for additional imaging, if any. The information about reports, findings, and recommendations is identified, captured, and securely transmitted by application engine to smart phone and web applications for patient care follow-up tracking. Using the system, the ordering or the referring doctor views the received reports, results and specific recommendations for additional imaging (RAI) if the report includes RAI. The application provides options to the referring doctor to act on the recommendations by accepting, declining, or deferring the recommendation and the selected option is tracked by the application in an audit log.

### PRIORITY CLAIM

This application is a continuation of U.S. patent application Ser. No. 16/296,181 filed Mar. 7, 2019 which claims the benefit of U.S. provisional patent application No. 62/639,996 filed Mar. 7, 2018, under 35 U.S.C. § 119, and the entire disclosure of each are incorporated herein by reference.

### TECHNICAL FIELD

The embodiments herein generally relates to web-based mobile application technologies and more particularly, relates to system and methods for reducing the rate of untracked follow-up recommendations for patient treatment, detecting and assessing the relevance of the recommendations, and reducing constraints on radiologists and referring providers tasks, and reducing time spent on results management in the areas of radiology and general health-care.

### BACKGROUND

Physicians struggle to track referrals and imaging results using manual methods or existing conventional technologies. An untracked referral may mean a test is never performed or performed too late for an optimal outcome. Untracked imaging results may mean other care is never performed or performed too late for an optimal outcome. These imaging results often include the radiologist's recommendation for additional imaging and the need for clinical correlation. Untracked follow-up recommendations will mean delayed diagnosis and intervention for some patients. ‘Untracked follow-up recommendation’ may represent a breakdown in communication or process between the referring physician or healthcare provider, the specialist such as a radiologist, and the patient. ‘Untracked follow-up recommendation’ may result, for example and without limitation, when a radiology report is never received by the referring physician, when a radiology report and recommendation are never read by the referring physician, when a recommendation in the radiology report is not clearly communicated to the referring physician, when a decision on the recommendation is deferred and the deferred status is never tracked, when an accepted recommendation, such as additional imaging is never ordered by a subsequent care giver, when an accepted recommendation is never communicated to the patient, when an accepted recommendation and ordered test is never scheduled by the patient, or when the patient never shows for the scheduled exam that was recommended by the radiologist and ordered by the referring physician.

The volume of imaging exams has grown very rapidly over the last 20 years. Imaging technology (the ability to see things on scans) has also evolved faster than the ability to know or understand the significance of what is seen. Radiologists often observe unexpected things (e.g. lung nodules) when a patient has a shoulder x-ray after an accident, where the purpose of the x-ray was to determine if a bone was fractured. Patient privacy laws and competing commercial interests among electronic medical record developers result in data loss. Doctors are specialized and paid to perform care for patients but are not paid to coordinate care by tracking follow-ups with the patient. The result of all of the above factors is that doctors now have a difficult time managing radiology test results because of both the sheer volume of tests and the volume of incidental findings in the tests. Positive test results (findings related to the reason for the exam) are managed reasonably well because medical treatment in response to the test result is often immediate and within the scope of practice of the doctor who ordered the test. Managing uncertain test results or unexpected test results incidental to the reason for the exam is much more difficult because of 1) the volume of such results, and 2) the lack of systems or tools to track these results to make sure the patient had the follow-up exam, especially when the care is best transferred to another doctor who will be ordering the follow-up exam. The reason so many of the recommended additional imaging tests never occur is because after the report with the recommendation is read, discussed with the patient, and filed into either a paper record or an electronic record of the treating doctor, it is then usually forgotten about either forever or until the next time the doctor sees the patient and reviews the medical record. There are no known systems for tracking these recommendations or incidental findings in ways that do not add to the doctor's workload. If the patient does not know about the recommendation, does not understand the recommendation, or forgets about the recommendation, and the treating doctor does not follow up with the patient, the radiologist's recommended follow-up imaging does not occur. The reason for this lack of follow-up is some combination of sheer volume, care follow-up not being a payable medical service for most doctors, and the fact that a recommendation may be for a health matter outside the expertise of the doctor who received the recommendation.

The US patent document Kulon “US20170109473” discloses a method and system that analyzes reports to identify patients who did not obtain recommended tests and, thereafter, sorts and filters the detected patients according to the relevance of the recommendation. Optionally, the systems and methods may discard recommendations of low relevance and facilitate the management of the detected patients using a human operator to efficiently supervise the system and manage tracking of the detected recommendations.

Providers have acknowledged the need to lower the rate of ‘Untracked follow-up recommendations’. However, the conventional solutions to date, such as EMR (Electronic Medical Records) alerts, emails, manual processes, and those described in Kulon, U.S. Pat. Pub. US20170109473 have not been well received because they add steps to the providers' workflows without fully ‘closing the loop’ on follow-up tracking. These systems and methods mentioned above “don't close the loop”, causing unnecessary harm to some patients, and result in related negative consequences associated with such unnecessary harm including, for example, higher overall health care costs, higher medical-legal liability costs for providers, patient dissatisfaction, and provider's (radiologist and referrer) dissatisfaction.

About twelve percent (12%) of imaging exams contain recommendations by the radiologist for additional imaging. The majority of the time—for example, about ninety percent (90%) of the time—these recommendations are accepted by the treating doctor or primary care provider. About twenty-six percent (26%) of the accepted recommendations do not result in the recommended additional imaging. These are the recommendations that are “lost to follow-up.” The report with the recommendation is read by the treating physician, discussed with the patient, and then filed into either a paper record or an electronic record of the treating doctor. Thereafter, the report with the recommendation is forgotten about either forever or until the next time the doctor sees the patient and reviews the medical record. This often results in loss of follow-up recommendations by the radiologist.

There are no known systems, applications, or methods for tracking these recommendations and incidental findings made by the radiologist. If the patient does not know about the recommendation, does not understand the recommendation, or forgets about the recommendation, and then the treating doctor or physician does not follow up with the patient, then the follow up care does not occur. The reason for this lack of follow-up is some combination of sheer volume, care follow-up not being a payable medical service for most doctors, and the fact that recommendation may be for a health matter outside the expertise of the doctor who received the recommendation. For example, a physician specializing in orthopaedic medicine receiving information or recommendation on presence of lung nodules may not focus on such information, as it does not pertain to his or her specialty or practice.

Hence, looking at the problems in the prior art and existing technologies, there is a significant need to prevent and minimize untracked follow-up recommendations for patient treatment.

The inventive concepts disclosed herein solve the problem of untracked follow-up recommendations for patients by providing a mobile application which reduces the rate of ‘Untracked follow-up recommendations, loosens the constraints on provider tasks, and reduces time spent on results management by connecting the radiologist or imaging facility to the treating doctor within the mobile application, managing test results and updating the patient EMR for both the radiologist and the treating doctor.

### SUMMARY

The invention overcomes the aforementioned problems by introducing a system and method for monitoring and capturing all medical reports such as radiology reports, in particular, those containing follow-up recommendations, reducing the rate of ‘Untracked follow-up recommendations,’ reducing the constraints on provider tasks, and reducing time spent on results management. The system and method is mainly comprised of an integration engine, a secure database with one or more radiology reports fed through the integration engine, a processor configured to execute the computer-readable instructions from memory to retrieve one or more radiology reports from a database and identify one or more instances of radiology information for the one or more radiology reports based on a preset configuration, and a phone or web application configured to receive one or more instances of radiology information to determine whether follow-up tracking is included in the one or more instances of radiology information and open a first loop based on the follow-up tracking included in the one or more instances of radiology information.

In an embodiment of the disclosed invention, the disclosed invention comprises a SPARC (Scalable Processor Architecture) application engine which reviews the medical report data, such as radiology report data, using a form of rules-driven processing to identify and capture key language elements contained in the report data. The SPARC application engine is programmed to identify, capture, and provide reports of any data element of the report. The radiology reports and addendums are fed through the integration engine into the secure database via HL7 (Health Level 7) feed or other available mechanisms such as web services or standard text files. The data elements to be reported by the SPARC application engine comprise, for example, the time when critical findings are communicated by the radiologists, who was notified of the finding, the presence of RAI (Recommendations for additional imaging), and the type of RAI.

In an embodiment of the disclosed invention, the disclosed invention claims that the radiology reports are organized into two groups: reports with RAI and reports without RAI. In addition to splitting radiology reports into two groups—those with and those without recommendations, the radloop application can also organize the reports with recommendations into customer-defined groups. The purpose of offering this functionality is to allow the referring doctor/navigator to organize recommendations in user-defined ways. User-specific organization of recommendations further reduces the result tracking effort, but tracking completeness is maintained. The reports are sent in real time to the mobile applications of a referring doctor or referrer. The referrer can be a physician such as an interventional radiologist, a primary care physician, or another specialist who may review these reports at any convenient time. For the group of reports with RAI, the mobile application provides functionality to allow the referrer to easily identify the test results and the specific RAI.

In another embodiment of the disclosed invention, the disclosed invention claims a method for monitoring all radiology reports and importantly those containing follow-up recommendations. The method is comprised of the steps of feeding of radiology reports and addendums through an integration engine into a secure database; identifying and capturing the RAI in radiology reports by the SPARC application engine; securely transmitting the reports to the mobile smart phone and web applications for follow-up tracking; organizing of reports into two group—reports with RAI and reports without RAI; identifying the test results and the specific RAI by the referrer for reports with RAI and closing the loop for reports without RAI; displaying of options in the application to either accept a recommendation, decline a recommendation, or defer the decision by the referrer upon reading the report and recommendation; ordering a recommended test or a different test by the referrer upon accepting the recommendation and closing of loop upon declining the recommendation; transmitting of these orders to a radiologist through the application and permitting the radiologist's and the referrer's offices to coordinate scheduling the follow-up exam wherein if the exam results include a new recommendation, a new loop opens upon introduction of a new recommendation in the follow-up exam results; and tracking the status of scheduling through exam completion.

In another embodiment of the disclosed invention, the disclosed invention claims the reasons for declining the recommendation, which includes for example and without limitation lack of clinical correlation, end of life or death, other patient decision, or referral to a another doctor who specializes in the proposed follow-up care. The mobile application continuously tracks the referrer's decisions and the status of each patient, and the referrer can reference additional information regarding the recommendation in the report.

In another embodiment of the disclosed invention, the disclosed invention claims a mobile application that allows for a secure call, text, or email with a consulting doctor. The referrer may have the ability to hit one button to immediately call either the patient or the radiologist or forward selected clinical information to a colleague with whom the referrer would like a consultation upon deferring of decision by the referrer. All deferred decisions are tracked inside the mobile application, enabling the referrer to periodically review them and, as appropriate, either further defer, accept, or decline the recommendation. The mobile application permits the referrer and radiologist to track the status of both existing test results and RAI.

In another embodiment of the disclosed invention, the disclosed invention discloses a quality measures reporting system of a smart phone or web-based application. The term “radloop” used herein refers to a trademark or service mark that is to be applicable with the underlying product directed to smart phone or web-based application pertaining to software related tools and applications. The “radloop” software based application includes specialized algorithms stored in memory or computer readable medium that are configured to be executed by at least one processor to perform a variety of specific, concrete and non-conventional steps that are directed to practical application of preventing untracked follow-up recommendations by a treating physician. The specialized algorithms are capable of being executed by at least one processor to provide a quality measure report feature with different modules involving a QC (Quality Control) review tool, ensuring correct capturing of findings by the SPARC engine; a master editor tool checking what the SPARC engine has found by measure; a critical finding review tool reviewing individual critical findings for a selected period of time; a report lookup tool locating any record by Unique ID (Identity Document); a generate submission report tool generating the required information to submit PQRS (Physician Quality Reporting System) or QCDR (Qualified Clinical Data Registry) data to a government agency; a reporting tool enabling the radiologist to report modality, date range based on practice defined parameters; an engine settings tool providing SPARC engine with the needed information to capture the various measure findings; a client tool capturing client information; a monitor tool providing real-time findings to the client by providing a feedback mechanism from the end user to the QC committee to discuss SPARC findings; a dashboard visualizing all data elements found in the mobile application; a dashboard scheduling tool simulating the mobile application environment from the client scheduler role; a dashboard practice simulating the mobile application environment from the practice scheduler role; an order CPT (Current Procedural Terminology) link tool linking the facility order catalog to the master CPT codes catalog so all CPT codes will flow to the referring provider office staff along with the facility order name for authorization purposes; and a measure CPT link facility adding and editing facility CPT codes.

The radloop application also standardizes the informal nomenclature of radiology tests used in the medical community. This standardization ensures the accurate communication of recommendations between radiologists and treating doctors. The radloop application accomplishes this standardization by converting the informal test names used by each radiology facility and each radiologist to the Current Procedural Terminology (CPT). CPTs, owned by the American Medical Association, are the accepted but unused US nomenclatures standard for all medical procedures. For example, Radiologist A recommends a follow-up CT of the abdomen on patient X. Radiologist B recommends a follow-up CT of the pelvis on patient Y. The relevant CPT in each case is a CT of the abdomen/pelvis. Radloop application communicates each of these recommendations from the radiologists to the treating doctor and also converts (maps) the recommendation to the actual, precise CPT. Now the treating doctor knows exactly which follow-up test is recommended. Radloop application then allows the treating doctor to order the correct test. Radloop also allows the treating doctor to order a different test. When a treating doctor orders a test using radloop application, he or she may only order a standard test. Now the radiology facility knows exactly which test to perform.

According to an example embodiment, the processor executes a specialized algorithm that receives information from a radiologist from an imaging facility and from a treating physician or doctor. The radiologist may enter his or her information via an electronic medical record application (EMR); the imaging facility may enter their information pertaining to the test results via their own EMR that is separate and distinct from the EMR used by the radiologist. And, finally, the treating physician or a primary care provider (“PCP”) may enter or retrieve information via his or her personal EMR that is separate and distinct from the EMRs operated by the radiologist and the imaging facility. One skilled in the art would appreciate that the information entered by these different entities may be different with respect to each other. For example, information entered by the treating physician or the PCP may be to have the patient undergo a particular test (i.e., scan or imaging) for a particular body part or region. This information is transmitted to the imaging facility, which views this information via its own EMR. After performing the requisite test ordered by the PCP, the imaging facility uploads the test results into its EMR. This information is then viewed by a radiologist who views the test results and provides his or her expert opinion via his or her EMR. In another embodiment when the imaging is performed in a hospital (as opposed to a radiologist's imaging center), the radiologist uses the hospital's EMR. In order to manage the flow of this information to and from different entities, via different devices, the processor of the radloop application manages theses test results, test orders, and expert opinions and continuously updates the respective EMRs.

The radloop application is embedded in these various EMRs of the respective entities such that it manages the flow of information between the EMRs. That way, a test order entered by a treating physician or PCP is received by an imaging facility, the test results prepared by the imaging facility are shared with an expert radiologist who interprets (reads) the images and issues a report, and the interpretation and opinion of the expert radiologist are communicated back to the treating physician who initially ordered the particular testing to be conducted.

The present inventive concepts overcome the drawbacks in the prior art by providing systems and methods in the form of a web-based mobile application that radiologists, primary care physicians and/or other referrers use to avoid untracked follow-up recommendations for patients, thereby avoiding delayed diagnosis for some patients, preventing unnecessary harm to the patients, and preventing related negative consequences associated with such unnecessary harm including higher overall health care costs, higher medical-legal liability costs for providers, patient dissatisfaction and provider dissatisfaction.

These and other aspects of the embodiments herein will be better appreciated and understood when considered in conjunction with the following description and the accompanying drawings. It should be understood, however, that the following descriptions, while indicating certain embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates schematic representation of a system to avoid untracked follow-up recommendations of patients using phone or web applications;

FIG. 2 illustrates schematic representation of a method to avoid untracked follow-up recommendations of patients using web applications;

FIG. 3 illustrates a radloop application with Quality measure reporting providing different tools;

FIG. 4 illustrates use of “Quality control review” radloop application tool;

FIG. 5 illustrates use of “Master Editor” radloop application tool;

FIG. 6 illustrates use of “Critical finding review” radloop application tool;

FIG. 7 illustrates use of “Report lookup” radloop application tool;

FIG. 8 illustrates use of “Generate submission Report” radloop application tool;

FIG. 9 illustrates use of “Reporting” radloop application tool;

FIG. 10 illustrates use of “Engine Settings” radloop application tool;

FIG. 11 illustrates use of “Client” radloop application tool;

FIG. 12 illustrates use of “Monitor tool” radloop application tool;

FIG. 13 illustrates use of “Dashboard” radloop application tool;

FIG. 14 illustrates use of “Dashboard scheduling” radloop application tool;

FIG. 15 illustrates use of “Dashboard practice” radloop application tool;

FIG. 16 illustrates use of “Order CPT link” radloop application tool; and

FIG. 17 illustrates use of “Measure CPT link” radloop application tool.

### DETAILED DESCRIPTION OF DISCLOSED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As used herein, computer program and/or software may include any sequence or human or machine-cognizable steps which perform a function. Such computer program and/or software may be rendered in any programming language or environment including, for example, C/C++, C#, Fortran, COBOL, MATLAB®, PASCAL, GO, RUST, SCALA, PYTHON™, assembly language, markup languages (e.g., HTML, SGML, XML, VoXML), and the like, as well as object-oriented environments such as the Common Object Request Broker Architecture (“CORBA”), JAVA™ (including J2ME, JavaBeans, etc.), Binary Runtime Environment (e.g., “BREW”), and the like.

As used herein, computer program and/or software may include any sequence or human or machine-cognizable steps which perform a function. Such computer program and/or software may be rendered in any programming language or environment including, for example, C/C++, C#, Fortran, COBOL, MATLAB™, PASCAL, GO, RUST, SCALA, Python, assembly language, markup languages (e.g., HTML, SGML, XML, VoXML), and the like, as well as object-oriented environments such as the Common Object Request Broker Architecture (“CORBA”), JAVA™ (including J2ME, Java Beans, etc.), Binary Runtime Environment (e.g., “BREW”), and the like.

As used herein, connection, link, and/or wireless link may include a causal link between any two or more entities (whether physical or logical/virtual), which enables information exchange between the entities. As used herein, computer and/or computing device may include, but are not limited to, personal computers (“PCs”) and minicomputers, whether desktop, laptop, or otherwise, mainframe computers, workstations, servers, personal digital assistants (“PDAs”), handheld computers, embedded computers, programmable logic devices, personal communicators, tablet computers, mobile devices, portable navigation aids, J2ME equipped devices, cellular telephones, smart phones, personal integrated communication or entertainment devices, and/or any other device capable of executing a set of instructions and processing an incoming data signal.

The volume of imaging exams have grown very rapidly over the last 20 years. Imaging technology (the ability to see things on scans) has also evolved faster than the ability to understand the significance of what is seen. Radiologists often observe unexpected things (e.g., lung nodules) in a shoulder x-ray after an accident, where the purpose of the x-ray was to determine if a bone was fractured. Patient privacy laws (HIPAA—Health Insurance Portability and Accountability Act) and competing commercial interests among electronic medical record developers result in data generation. Since doctors are specialists, for example, a specific specialist treating fractured shoulders in an emergency room, the doctors have no interest, training or experience in treating lung nodules. Such physicians are only paid to and responsible for performing the care of fixing or repairing the fractured shoulder, and not coordinating care of the patient with respect to tracking that the patient follows up with a cancer specialist, for example.

The result of all of the above factors is that doctors now have a difficult time managing radiology test results because of both the sheer volume of tests and the volume of incidental findings in the tests. Positive test results (Did the patient fracture her shoulder? Yes, the patient fractured her shoulder) are managed reasonably well because medical treatment in response to the test result is often immediate and within the scope of practice of the doctor who ordered the test. Managing uncertain test results (the mammogram shows something, let's do an ultrasound or a MRI to better see the something) or unexpected test results (the lung nodule during a shoulder x-ray) is much more difficult because of 1) the volume of such results and 2) the lack of systems or tools to use to track these results (to make sure the patient had the follow-up exam, especially when the follow-up exam would be ordered by a different doctor)

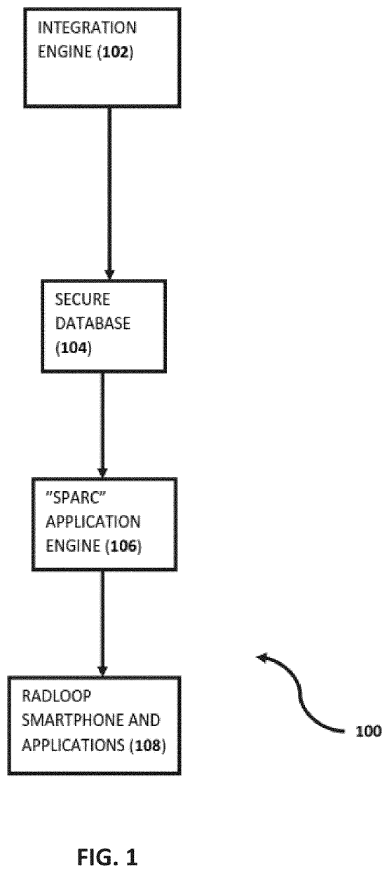

FIG. 1 illustrates an architectural framework of a system (100) to prevent untracked follow-up recommendations of patients using the radloop smart phone or web applications (108). The system (100) stores all radiology reports in a secure database (104) via an integration engine (102). The system (100) uses an HL7 feed mechanism or various other mechanisms such as web services or standard text files to feed all radiology reports into the secure database (104). The report data is reviewed by a SPARC application engine (106). The SPARC application engine (106) is programmed to identify, capture and report on any data element of a radiology report, such as the time when critical findings are communicated by the radiologists, who were notified of the finding, as well as the presence and type of RAI (Radiologist recommendation for additional imaging). The SPARC application engine, (106) after identifying and capturing the RAI, securely transmits this information to smart phone and web applications (108) for follow-up tracking.

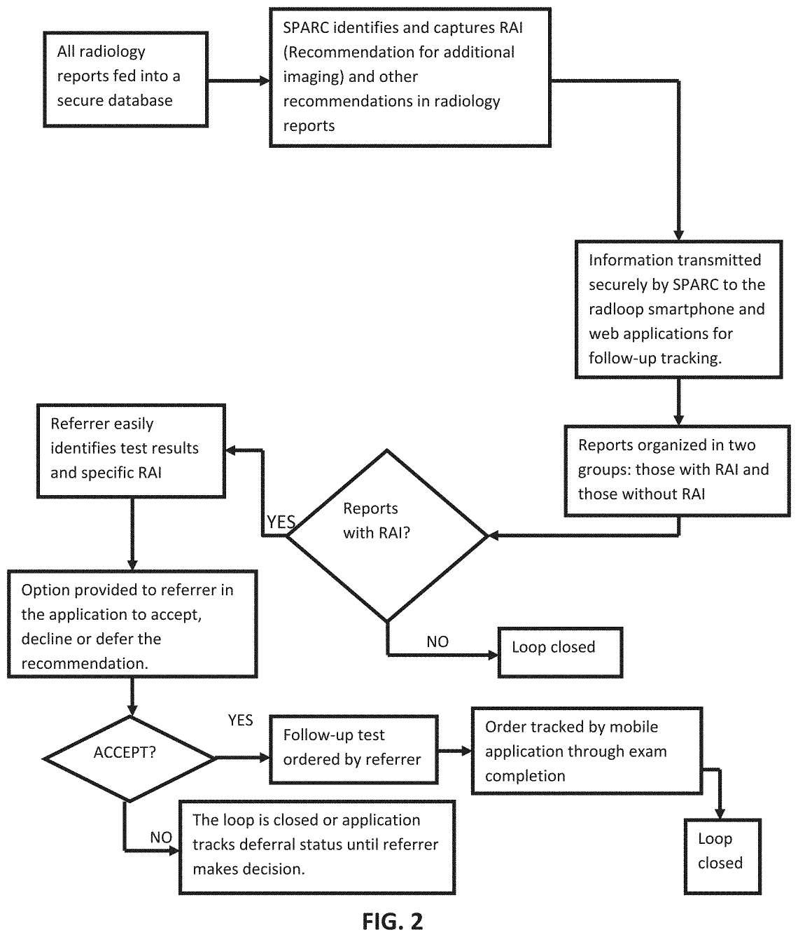

FIG. 2 illustrates a method to avoid untracked follow-up recommendations of patients. All radiology reports are stored in a secure database (104) by using an HL7 feed or other mechanisms via an integration engine (102). The report data is analysed by an application engine “SPARC” (106) which in turn identifies and captures RAI and other recommendations in radiology reports and then securely transmits information to smart phone and web applications (108) for follow-up tracking. Based on information provided by SPARC application engine (106), the reports are organized into two groups, one with RAI and one without RAI. In addition to splitting radiology reports into two groups—those with and those without recommendations, the radloop application can also organize the reports with recommendations into customer-defined groups. The purpose of offering this functionality is to allow the referring doctor/navigator to organize recommendations in user-defined ways. User-specific organization of recommendations further reduces the result tracking effort, but tracking completeness is maintained. The physicians who refer to the radiologist (or the hospital) receive the radiologist report including any RAI on the smart phone or web applications (108), and their group practices receive the same data on the smart phone or web applications (108). Referrers see only data of their patients or their practice patients. The reports are sent in real time to the smart phone or web applications (108). Upon reading the report and the recommendation, the referrer has the option, in the smart phone or web applications (108), to immediately accept or decline the recommendation, or defer the decision. The smart phone or web applications (108) continuously track the referrer's decisions and the status of each patient. If the referrer accepts the recommendation, the referrer may immediately and easily, inside the smart phone or web applications (108), order the recommended test. The referrer may instead immediately order a different test. The smart phone or web applications (108) transmit these orders automatically to the radiologist. The smart phone or web applications (108) then permit the radiologist's and the referrer's offices to coordinate scheduling the follow-up exam and tracks the status of scheduling through exam completion. If the referrer declines the recommendation, the referrer may select the reason for declining the recommendation. Reasons for declining a recommendation can include lack of clinical correlation, end of life or death, other patient decision, or referral to a specialist. The decision to decline closes the loop on the recommendation for that referrer. The referrer may want more information before accepting or declining a recommendation and select the defer option. Inside the smart phone or web applications (108), the referrer is given the ability to hit one button to immediately call either the patient or the radiologist or to forward selected clinical information to a colleague with whom the referrer would like a consultation. The smart phone or web applications (108) allow for a secure call, text, or email with the consulting doctor. All deferred decisions are tracked as such inside the radloop application (108), enabling the referrer to periodically review them and, as appropriate, either further defer, accept, or decline the recommendation. The radloop application (108) permits the referrer and radiologist to track the status of both all test results and recommendations for additional imaging in a highly efficient and cost-effective manner. Recommendations for additional imaging are then tracked through all stages (the referrer's decision, the patient's decision, the scheduling of the test, and test completion) to eliminate the failure to perform follow-up testing due to some break in communication or other failure to track the recommendation. The radloop application (108) documents all decisions and events made by the radiologists, referrer, and patient. The radloop application (108) tracks all recommendations for additional imaging made by the radiologist. If the referrer declines a recommendation, the loop is closed. If the referrer accepts a recommendation, the referrer orders the follow-up test, and the radloop application (108) tracks that order through exam completion. Upon exam completion, the loop is closed. If the exam results include a new recommendation, a new loop opens. If a patient won't schedule an exam, doesn't show up for a scheduled exam, or goes to a different imaging facility, the radloop application (108) documents those patient decisions, and the loop is closed for the referrer and original radiologist. When the referrer defers making the decision to accept or decline a recommendation, the radloop application (108) tracks this deferral status until the referrer makes the decision. The radloop application, being installed on a mobile application or a computing device, is configured to perform and execute a series of steps. In particular, a processor or controller is configured to execute computer-readable instructions to automatically capture all recommendations for additional imaging stated in an imaging report. The processor or controller is further configured to execute the computer-readable instructions to automatically track those recommendations made by the specialist, for example a radiologist, until follow-up imaging is performed or until an alternative reason is provided. Alternative reasons include the following: there are no follow-up recommendations, the patient on whom the additional imaging was to be performed has died, the patient is in hospice (end of life care), the patient had imaging done somewhere else at an alternative site, or the patient refused the imaging.

The processor or controller is further configured to execute the computer-readable instructions to track the activities of a referring doctor who is primarily responsible for tracking the recommendation and allowing that doctor to transfer responsibility to another doctor or treating physician in the event the primary or first doctor is 1) unqualified to continue care (for example, an orthopaedist must transfer care to a pulmonologist), 2) going off shift in a hospital emergency department or inpatient unit, or 3) otherwise becomes unavailable or out of office for any given reason. The processor is also further configured to execute computer-readable instructions which allow the second doctor to accept or decline this transfer of responsibility.

In the event that a treating physician does not have the application or system for tracking the imaging results and treatment plan of the patient, the application or system includes a navigator function. The processor or controller is further configured to execute the computer-readable instructions to have the navigator function allow all of the aforementioned steps to be tracked inside the radloop application and, accordingly, communicate with the imaging facility entity or hospital. This navigator functionality permits an employee of an imaging facility or hospital to track follow up recommendations inside the application when the referring doctor does not use the radloop application. Additionally, in an embodiment the navigator function allows an employee of the hospital to transfer both the test status and responsibility of care to doctors within and outside the particular facility. For example and without limitation, Dr. X in the hospital emergency department orders a CT on a patient presenting with abdominal pain. Before the CT is performed and interpreted by the expert radiologist, Dr. X's shift ends and Dr. Y's shift begins. The navigator function allows the navigator employee to transfer all of Dr. X's open cases to Dr. Y. Similarly, in the inpatient setting, hospitalist doctors work shifts, and the navigator function transfers responsibility of care as those shifts change. This transfer of care inside the radloop application during shift changes ensures that imaging test results may be acted on immediately by the doctor on duty even though he/she wasn't the ordering doctor for the test and also ensures that the hospital can more easily follow up on additional recommended testing until the loop is closed for that patient. The navigator may also track and transfer test results to an appropriate doctor outside the facility, when the follow up testing won't be ordered by the facility doctors (for example, emergency department patient was in a car accident and X-rays ordered to look for bone fractures reveal lung nodules).

The processor or controller is further configured to execute the computer-readable instructions to track all the recommendations provided by a specialist, such as a radiologist or a primary physician, and also organize all the outstanding recommendations to make them easier for the treating physician (through the navigator function) to focus on the next steps in patient care and treatment. The next steps may correspond to activity that the controller or processor characterizes as a follow-up. These may be, for example, calling the patient, calling a consulting doctor, transferring the responsibility for care to another doctor, ordering the recommended test, or receiving the new test results. The processor or controller is further configured to execute the computer-readable instructions to repeat this above process or, in turn, close the loop in the event the alternative reasons are provided. The alternative reasons include, as noted above, the patient on whom the additional imaging is to be performed has died, the patient is in hospice (end of life care), the patient had imaging done somewhere else at an alternative site, or the patient refused the imaging.

One skilled in the art would appreciate that the processor or controller is configured to execute the computer-readable instructions to perform the aforementioned steps of tracking recommendations and following through with additional tasks without resulting in any additional steps to the work efforts of the radiologists, treating physicians, or other specialists. The processor controller is specialized to perform these tasks in a practical application that improves on the archaic electronic medical records (EMR) that are generally disliked by physicians as the EMRs are hard to work with, they add additional work for the treating physicians, and they do not provide pertinent information that is readily accessible to the treating physician.

In interpreting the reports and recommendations provided by specialists such as radiologists, the processor or controller is configured to execute the computer-readable instructions to identify, interpret, and comprehend the jargon or language used by the treating physician and then convert the same to standard nomenclature. In other words, the processor or controller standardizes the informal nomenclature of radiology tests used in the medical community. This standardization ensures the accurate communication of recommendations between radiologists and treating doctors. The controller or processor is configured to execute the computer-readable instructions to convert the informal test names used by each radiology facility, for example, and each radiologist to the Current Procedural Terminology (CPTs). CPTs, owned by the American Medical Association, are the accepted but unused U.S. nomenclature standard for all medical procedures. Each CPT has a 5-digit number.

For example, radiologist A recommends a follow-up CT scan of the abdomen on patient X. Radiologist B recommends a follow-up CT scan of the pelvis on patient Y. The relevant CPT in each case is a CT of the abdomen/pelvis. The controller or processor is configured to execute computer-readable instructions to communicate each of these recommendations of the radiologists to the treating doctor, and it also converts, or maps, the recommendation to the actual, precise CPT. Now the treating doctor knows exactly which follow-up test is recommended for which particular patient. The controller or processor is further configured to execute the computer instructions to receive input from the treating doctor to order this correct test, in turn, avoiding any guessing or estimating by the treating physician as to what test to order as part of the patient's treatment plan. Alternatively, the treating physician may order a different test for the patient. Such communication is passed onto the imaging facility, or alternative site, where testing is to be performed.

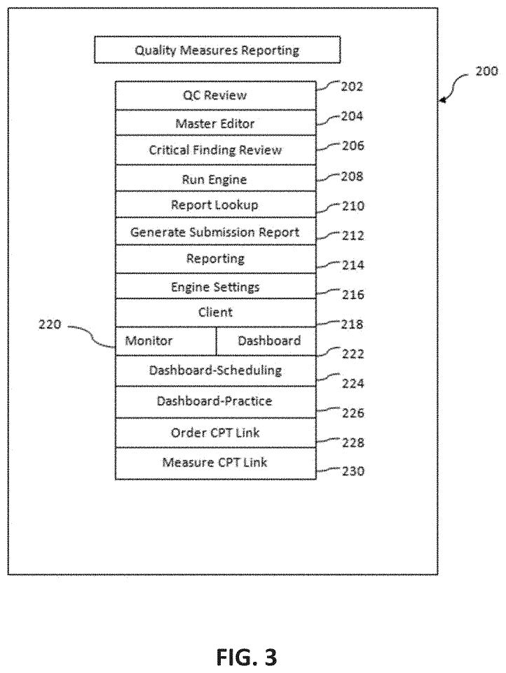

FIG. 3 illustrates a radloop application (108) with Quality measure reporting (200) providing different modules. The different modules disclosed herein are QC (Quality Control) review (202), Master editor (204), Critical finding review (206), Run engine (208), Report lookup (210), Generate submission report (212), Reporting (214), Engine settings (216), Client order (218), Monitor (220), Dashboard (222), Dashboard-scheduling (224), Dashboard-practice (226), Order CPT link (228) and Measure CPT link (230). The radloop application (108) standardizes the informal nomenclature of radiology tests used in the medical community. This standardization ensures the accurate communication of recommendations between radiologists and treating doctors. The radloop application (108) accomplishes this standardization by converting the informal test names used by each radiology facility and each radiologist to the Current Procedural Terminology as noted in the above example. CPTs, owned by the American Medical Association, are the accepted but unused US nomenclatures standard for all medical procedures.

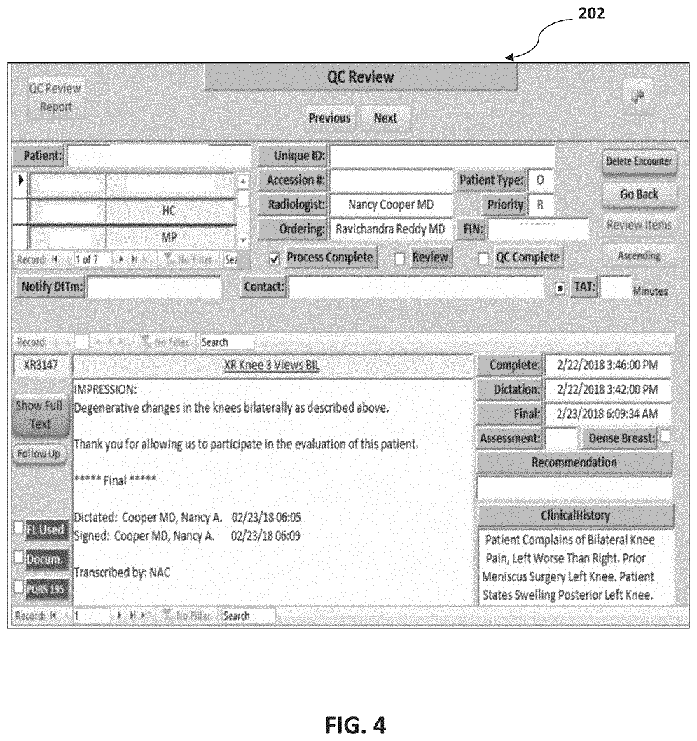

FIG. 4 illustrates “QC review” (202) which is a radloop application (108) tool providing quality review of each radiologist's finalized report. It ensures “SPARC” (106) is capturing the findings correctly. This view provides access to all needed information to assess the report. This also allows capture of the QC (Quality control) rate for reporting purposes. It has the ability to mark for further review if the radiologist, physician and/or another specialist need to consult with the QC team for questionable findings. A User can toggle between the full text of the report and the impression only.

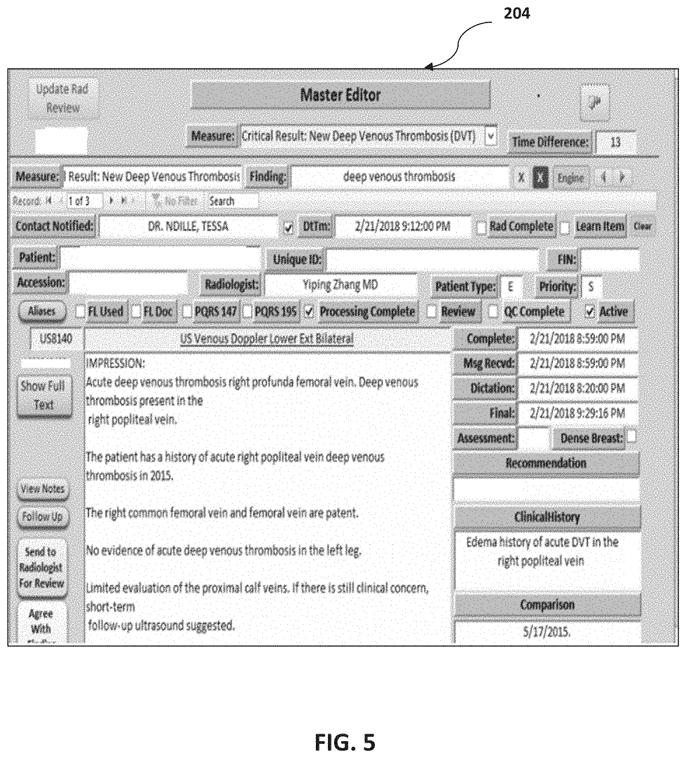

FIG. 5 illustrates “Master Editor” (204) which is a radloop application (108) tool to review what SPARC (106) has found by measure. Measures are simply definitions for SPARC (106) to look for. A measure can be a quality control item, critical finding, recommendations, or anything else found in the report text. The QC analyst may have the ability to mark items to be reviewed by the QC committee for questionable findings. If SPARC (106) has found something that is not relevant, the QC analyst can add the information to SPARC's (106) negative findings database so that SPARC (106) skips the finding in the future. To test if the engine modification is successful, the QC analyst can mark the report to be re-evaluated by SPARC (106) in real-time. If an interesting or unique finding is noted, the report can be marked as a “learn item” to be reported on later. This information can be used to help the radiologist better understand what may be needed to ensure SPARC (106) can capture the finding or identify areas of improvement. The examples of measures that can be found by SPARC (106) are ACRad 5 abnormal interpretation rate, ACRad abnormal screening mammogram, critical results protocols, critical result such as new DVT (Deep Venous Thrombosis), critical test protocols, follow-up exam obtained, PQRS measures including PQRS 145 for Fluoroscopy (FL) images and time documented, PQRS 147 for nuclear medicine correlation with existing image and PQRS 195 for stenosis measurement in carotid reports, QMR (Quick medical reference) critical finding involving radiologist practice critical finding policy, QMR follow-up obtained, QMR follow-up recommended, QMR important finding, QMR incidental finding, QMR STAT finding, QMR urgent finding, result requiring follow-up protocol, unexpected result protocol, urgent result protocol and wet reading identifier.

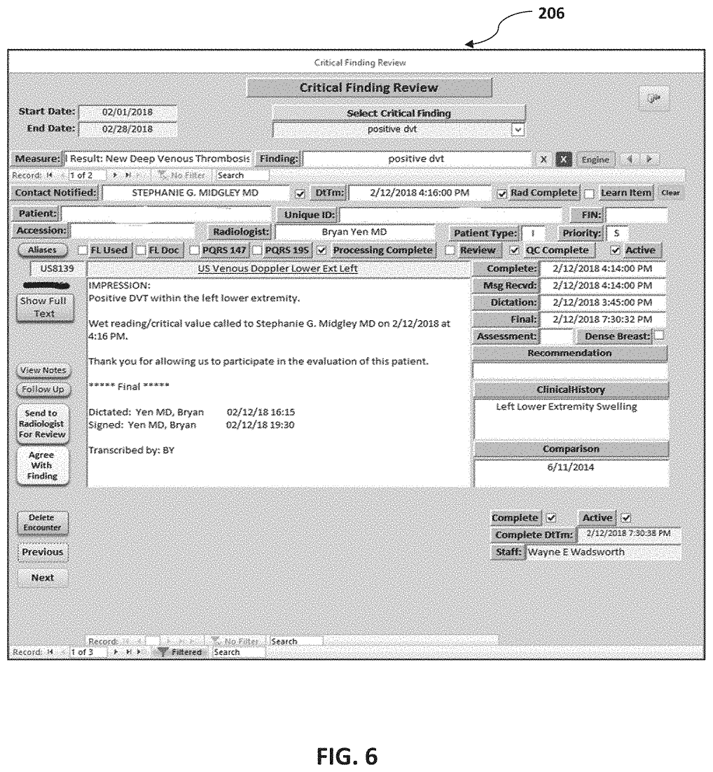

FIG. 6 illustrates “Critical finding review” (206), a radloop application (108) tool to review individual critical findings for a selected period of time. As new measures are added, SPARC (106) will need to review several reports to ensure capture of the findings. This tool allows the QC analyst to focus on the finding to determine what changes are needed to be made in SPARC (106). It also has ability to quantify data results based on date of service range.

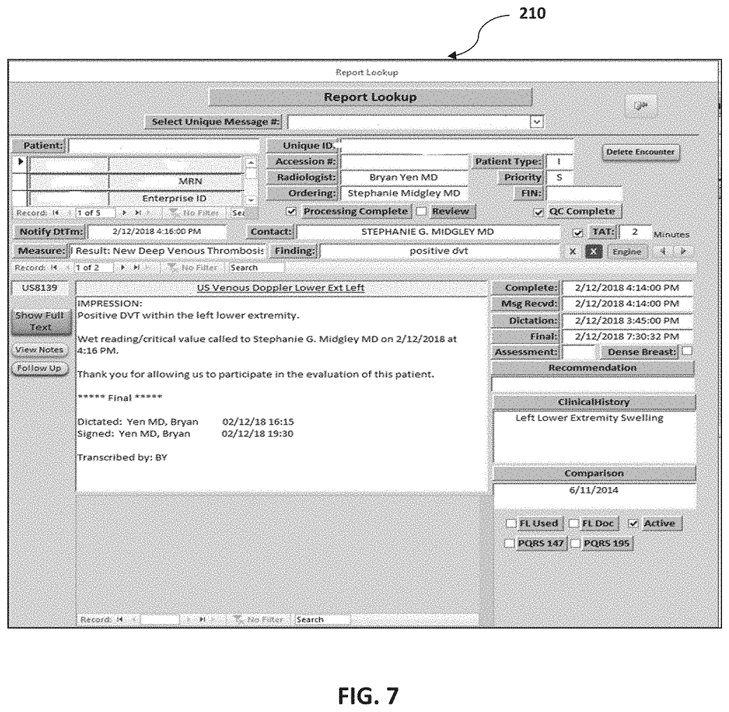

FIG. 7 illustrates “Report lookup” (210), a radloop application (108) tool to locate any record by Unique ID. All data can be modified by the QC analyst if needed using this tool.

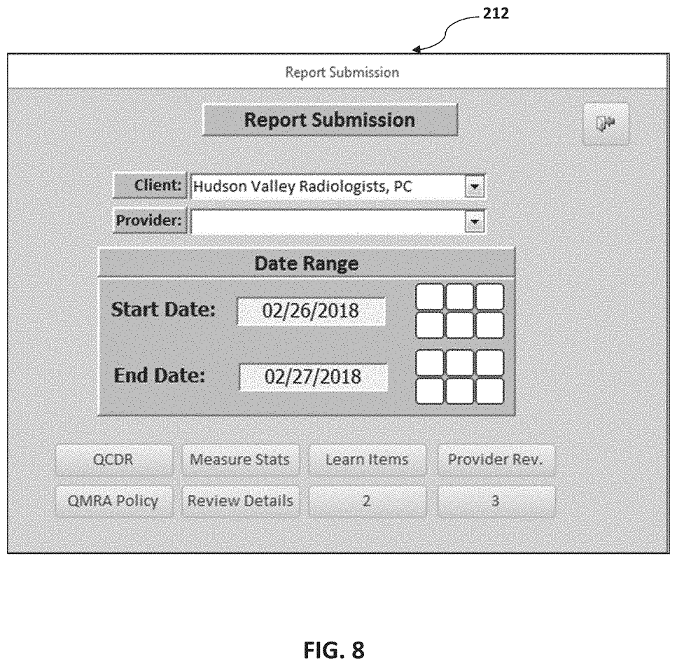

FIG. 8 illustrates “Generate submission Report” (212) radloop application (108) tool to generate the required information to submit PQRS or QCDR data to government agency. Reports can be generated to provide feedback to the radiologists for any timeframe. This provides continuing education on the findings.

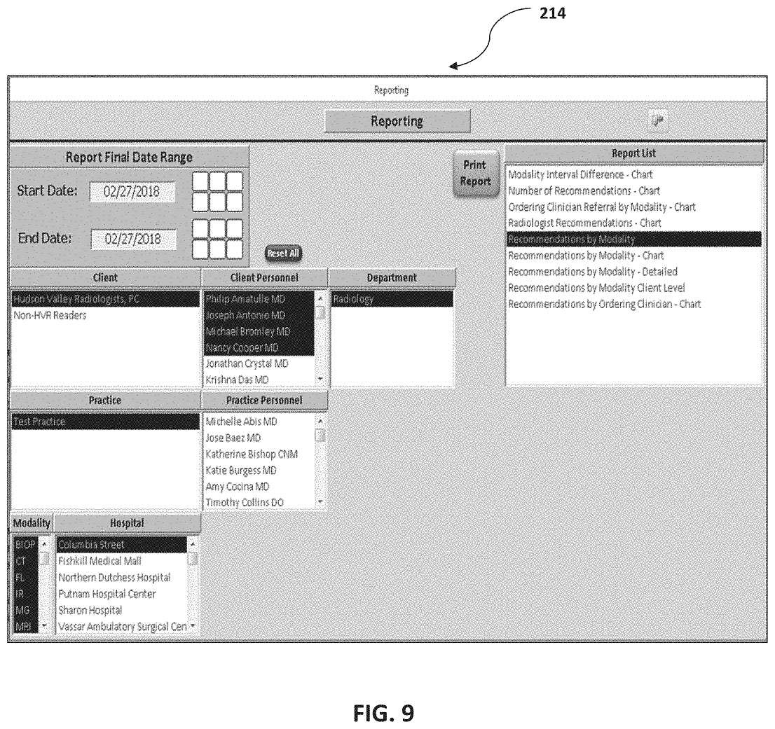

FIG. 9 illustrates “Reporting” (214), a radloop application (108) tool to report by radiologist, modality, and date range based on practice defined parameters.

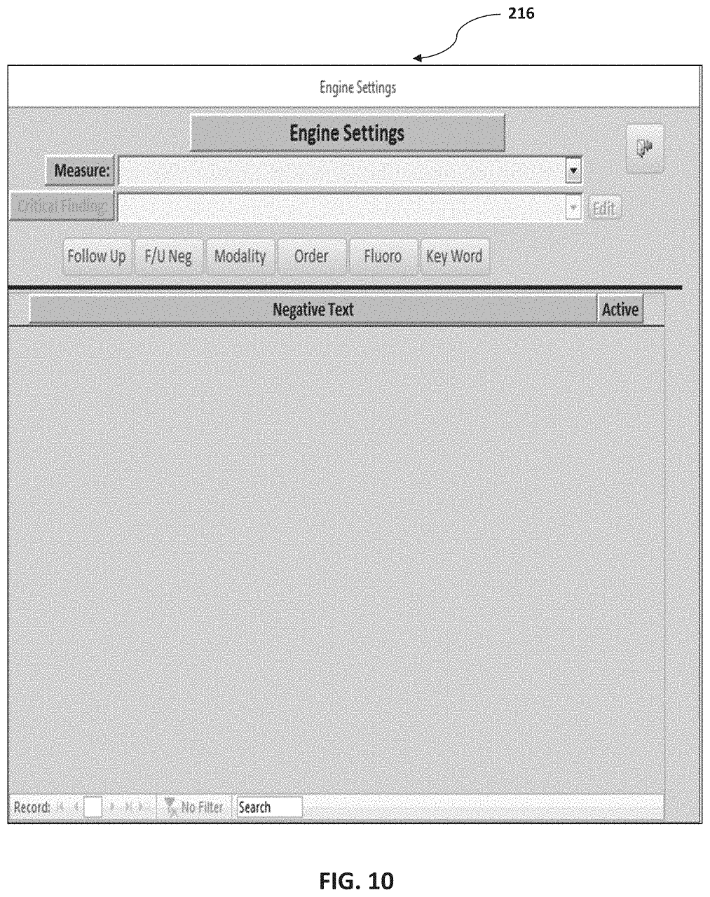

FIG. 10 illustrates “Engine Settings” (216) radloop application (108) tool to provide SPARC (106) with the needed information to capture the various measure findings. There are various tools used under engine settings tool, examples of which are as follows: a) “Follow up” tool identifies the intervals to be extracted from the report text, b) “F/U neg” (Follow up negative) tool is used to remove all unwanted text from the reports so SPARC can properly identify follow-up recommendations, c) “Modality” tool identifies the text needed to find each modality within the report, d) “Order” tool identifies the text needed to find each order within the report, e) “Fluoroscopy” search text tool identifies the text needed to find reports which contain information about fluoroscopy performed, f) “Keyword” tool identifies text needed to pull the notification information found in the report and g) “Connection search text” tool identifies connection words to enable SPARC to pull multiple recommendations within a single sentence.

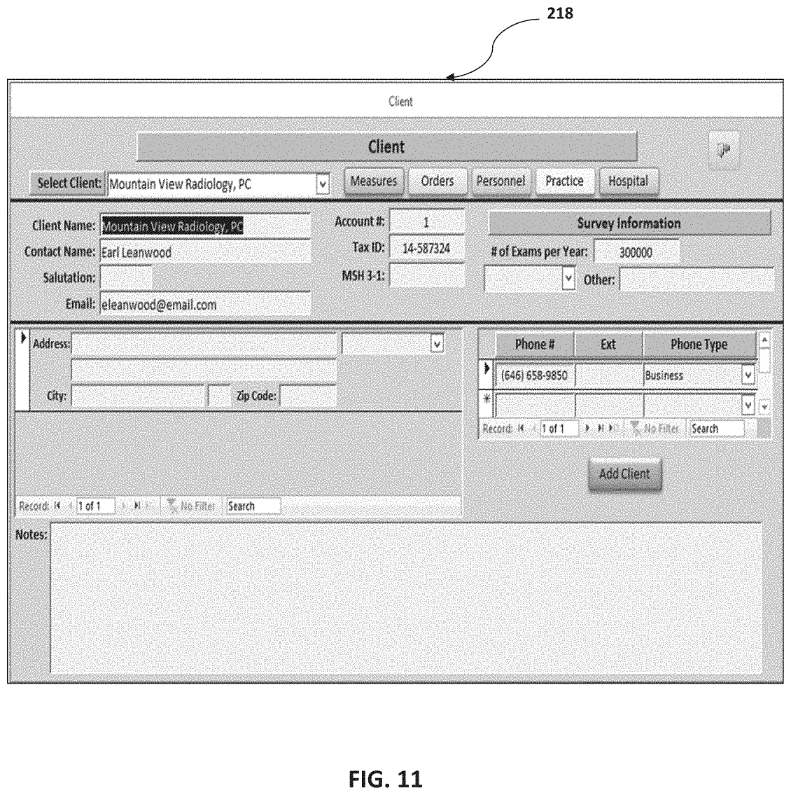

FIG. 11 illustrates “Client order” (218) tool to capture the client information. There are various tools used under client tool which are as follows: a) “Client Measure” tool is used to identify what measures the client wants to look for within their reports, b) “Client order” tool (218) is used to link a standard name, modality, body part, contrast, laterality, and CPT codes, identify critical test orders, identify screening mammography orders, identify biopsy orders, identify orders which should always be considered immediate orders, identify if the order should have a BIRAD (Breast imaging reporting and data system) indicated in the report, c) “Client personnel” identify the radiologist, scheduler, and administrator for the client, d) “Client practice” identifies the referring physician practices that would like to participate, and e) “Client hospital” identifies hospital units associated with the radiology group for reporting purposes.

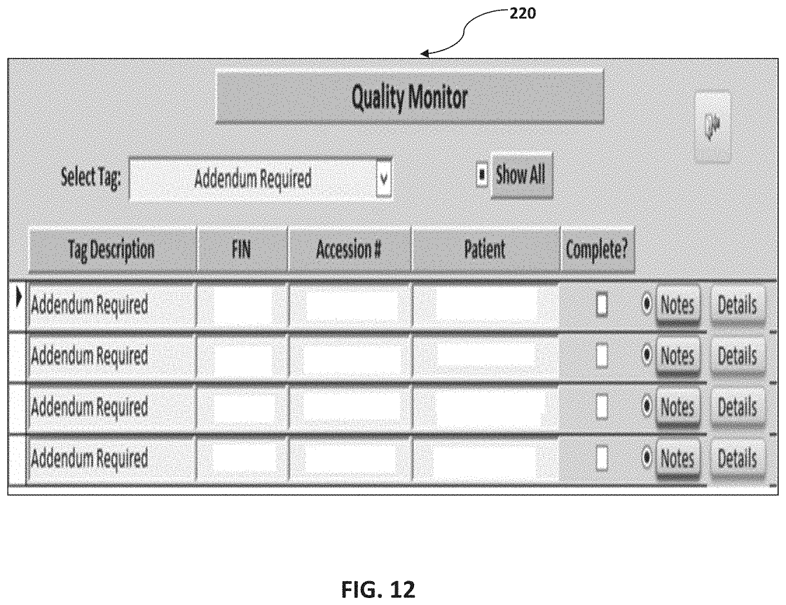

FIG. 12 illustrates “Monitor tool” (220) radloop application (108) tool to review what SPARC (106) has found by measure. The monitor tool provides real-time findings to the client. The monitor provides a feedback mechanism from the end user to the QC committee to discuss SPARC (106) findings. The examples of monitors used may include, for example, but not limited to, Addendums Required, Complication Review, Discrepancy Review, Follow Up Recommended, Laterality Review, Pathology Review, Pathology Overdue, Radiologist Review, Repeat Symptom Review, Stat Follow Up and Stat Order Not Final. The notes are used to communicate between the QC analyst, QC committee, and the end users.

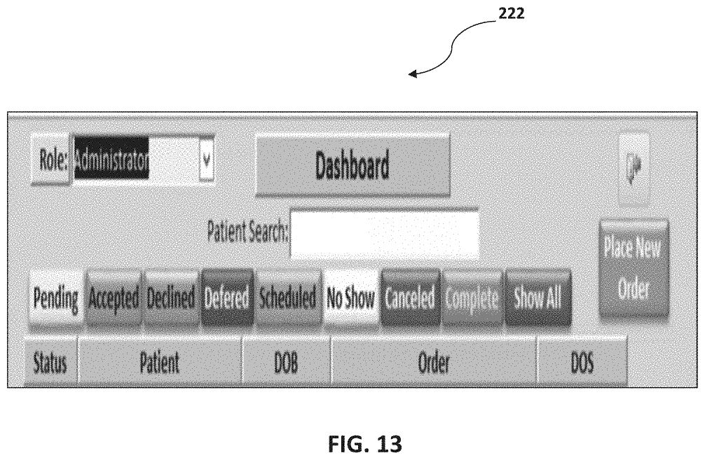

FIG. 13 illustrates “Dashboard” (222) radloop application (108) tool used to visualize all data elements found in radloop application (108). This is also an administrator's view to allow all adjustments to be made as needed.

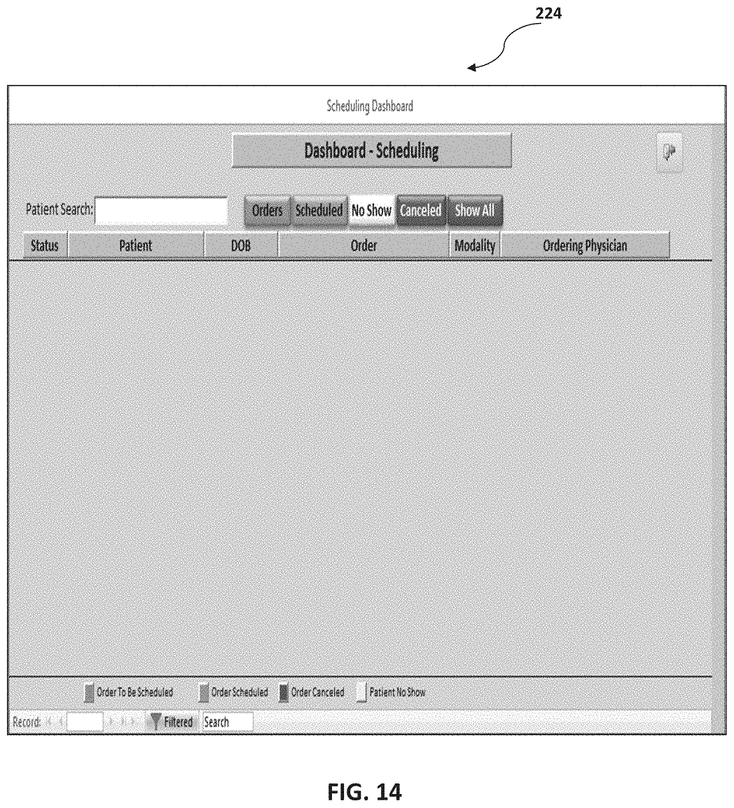

FIG. 14 illustrates “Dashboard scheduling tool” (224) radloop application (108) tool used to simulate the radloop application (108) environment from the client scheduler role.

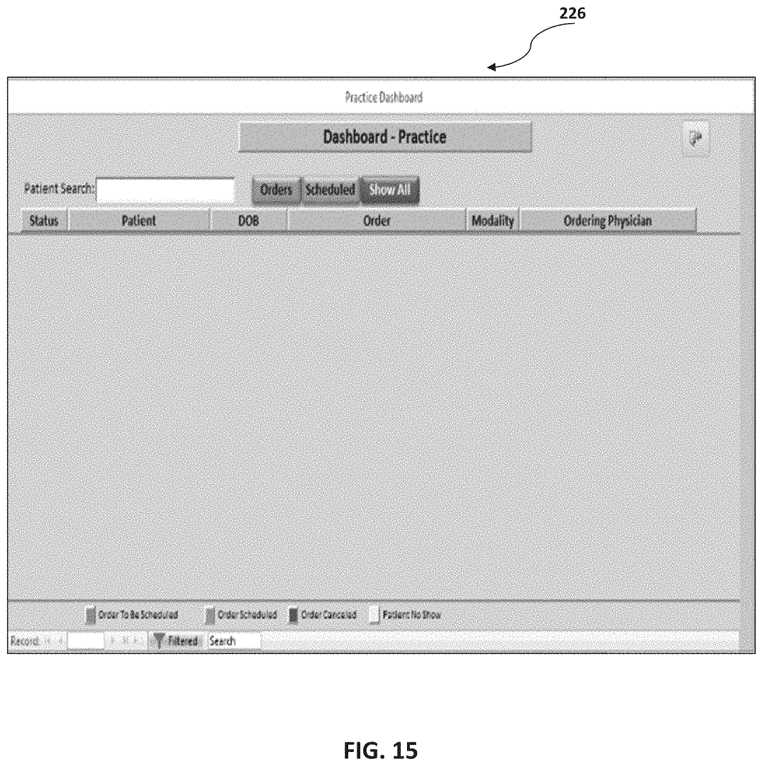

FIG. 15 illustrates “Dashboard practice” (226) radloop application (108) tool used to simulate the radloop application (108) environment from the practice scheduler role.

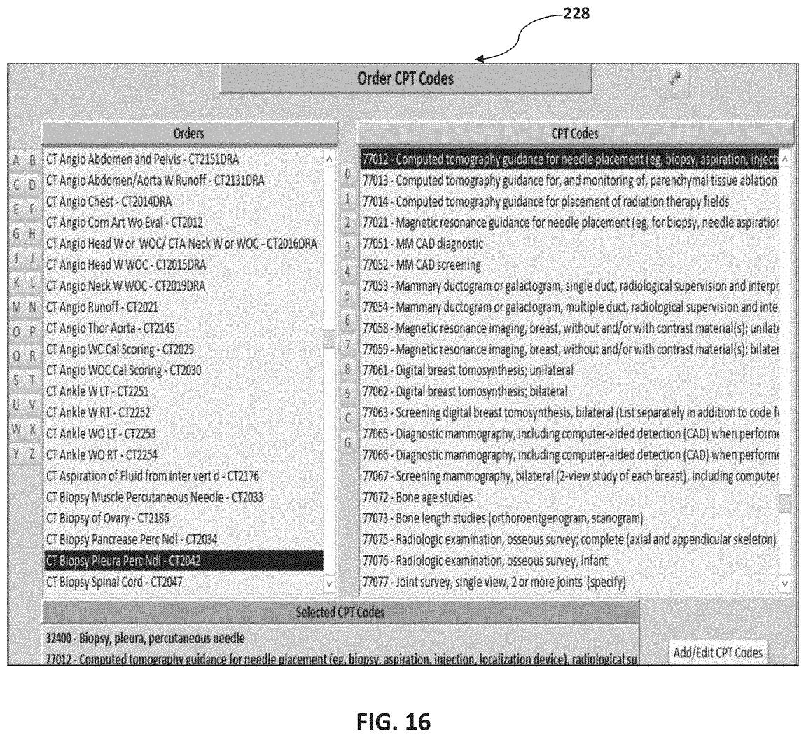

FIG. 16 illustrates “Order CPT link” (228) radloop application (108) tool to link facility order catalogue to master CPT codes catalogue so that all CPT codes will flow to the referring provider office staff along with the facility order name for authorization purposes.

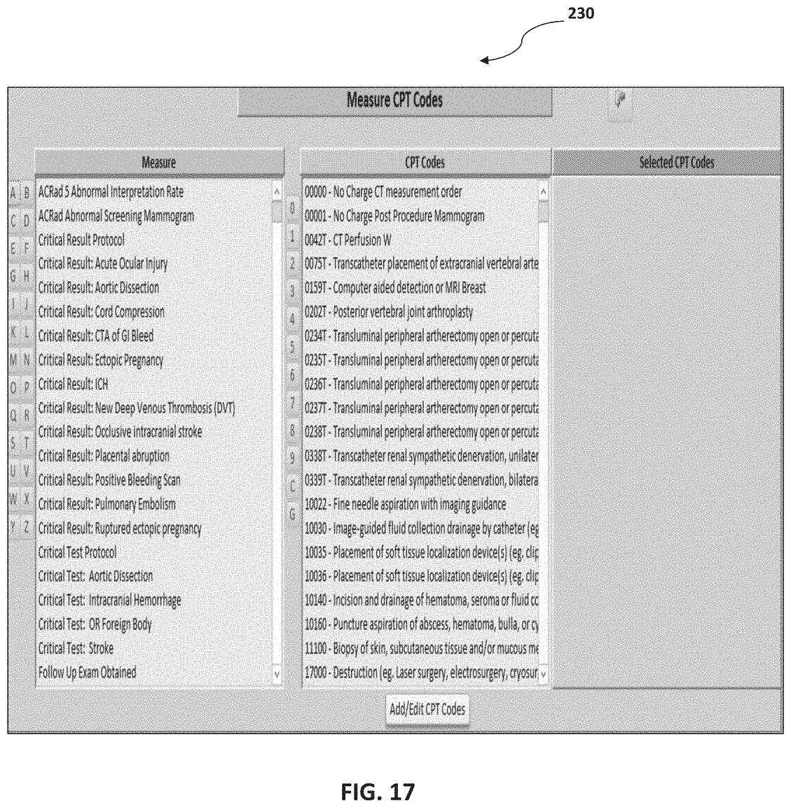

FIG. 17 illustrates “Measure CPT link” (230) tool. It adds facility measures and edits CPT codes.

The foregoing description of the specific embodiments will so fully reveal the general nature of the embodiments herein that others can, by applying current knowledge, readily modify and/or adapt such specific embodiments for various applications without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Those skilled in the art will recognize that the embodiments herein can be practiced with modification within the spirit and scope of the appended claims.

What is claimed is:

1. A system for managing radiology reports and addendums including those containing follow-up recommendations, comprising:
a memory having computer readable instructions stored thereon;
an integration engine;
a secure database with one or more radiology reports fed through the integration engine;
a processor configured to execute the computer-readable instructions from the memory to retrieve one or more radiology reports from the secure database, assess one or more instances of radiology information for the one or more radiology reports based on a preset configuration and determine a referrer associated with the one or more radiology reports; and
a smart phone or web application configured to receive the one or more instances of radiology information to determine whether a follow-up tracking is included in the one or more instances of radiology information, open a first loop based on the follow-up tracking included in the one or more instances of radiology information, report to the referrer the one or more instances of radiology information associated with the one or more radiology reports and display options in the smart phone and web application for the referrer to act on the recommendation by either accepting a recommendation, declining a recommendation, or deferring a decision or to first consult with a patient or another doctor or transfer responsibility to another doctor.

2. The system as claimed in claim 1, wherein the processor is a scalable processor architecture (SPARC) application engine which reviews the report data using rules driven processing to identify and capture key language elements contained in the report data.

3. The system as claimed in claim 2, wherein the SPARC application engine is programmed to identify, capture, assess, and report on any data element in the report.

4. The system as claimed in claim 3, wherein the data elements to be assessed and reported by the SPARC application engine comprise a time when actionable findings are communicated by a radiologist, who was notified of the finding, a presence of recommended additional imaging, and type of recommended additional imaging (RAI).

5. The system as claimed in claim 1, wherein the processor is further configured to execute the computer-readable instructions from the memory to organize the radiology reports into groups of reports with RAI and reports without RAI.

6. The system as claimed in claim 1, wherein the smart phone or web mobile application provides functionality to allow the referrer to easily identify a test result and a specific RAI for the group of reports with RAI.

7. The system of claim 6 wherein the at least one processor is further configured to execute the computer-readable instructions to manage completed radiology tests, manage orders for additional radiology tests, track an opinion of at least one doctor regarding results of the radiology test, and continuously update at least one patient EMR.

8. The system as claimed in claim 1, wherein the radiology reports and addendums are fed through the integration engine into the secure database via an HL7 feed.

9. A method for managing radiology reports and addendums including those containing recommendations for additional imaging (RAI), the method comprising:
feeding one or more radiology reports and addendums through an integration engine into a secure database;
identifying and capturing a RAI in radiology reports by a scalable processor architecture (SPARC) application engine;
identifying and capturing a referrer in radiology reports by the SPARC application engine;
securely transmitting the reports to a smart phone and web application for follow-up tracking;
organizing of the reports into groups of those reports with RAI and those without RAI;
identifying test results and specific RAI for the reports with RAI and closing of loop for reports without RAI;
reporting to the referrer the one or more radiology reports and addendums organized into groups of those reports with RAI and those without RAI;
displaying of options in the smart phone and web application for the referrer to either accept a recommendation, decline a recommendation, or defer a decision;
transmitting of orders for follow-up testing to a radiologist through the application; and
permitting the radiologist's office and the referrer's office to coordinate scheduling a follow-up exam and tracking a status of scheduling through exam completion.

10. The method as claimed in claim 9 wherein reasons for declining the recommendation includes lack of clinical correlation, end of life, other patient decision, or referral to a specialist.

11. The method as claimed in claim 9, wherein the referrer can be a radiologist, a primary care physician, or another specialist.

12. The method as claimed in claim 9, wherein the application continuously tracks a referrer's decisions and a status of each patient.

13. The method as claimed in claim 9, wherein the application allows for a secure call, text, or email with a consulting doctor.

14. The method as claimed in claim 9, wherein all deferred decisions are tracked inside the application, enabling the referrer to periodically review them and, as appropriate, either further defer, accept, or decline the recommendation.

15. The method as claimed in claim 9, wherein the application permits the referrer and radiologist to track a status of both existing test results and RAI.

16. The method as claimed in claim 9, wherein if an exam result includes a new recommendation, a new loop opens upon introduction of the new recommendation in the exam result.

17. The method of claim 8 further comprising continuously updating at least one EMR.

18. A system for tracking follow-up recommendations for a patient, comprising:
a memory having computer-readable instructions stored thereon;
at least one processor configured to execute the computer-readable instructions to:
initiate a loop based on receiving an input from a first physician, the loop being opened based on the receipt of the input wherein the input includes test results, and wherein the first physician is capable of interpreting the test results;
transmit the results to a second physician;
receive interpretation of the results from the first physician;
transmit the interpretation of the results to the second physician;
scan the interpretation of the results for additional information, the additional information corresponding to a follow-up recommendation;
display options in a smart phone and web application for the second physician to either accept a recommendation, decline a recommendation, or defer a decision and
initiate the loop to be closed only if the additional information indicates an event corresponding to completing treatment plan of the patient.

19. The system of claim 18, wherein the first physician is a specialist such as a radiologist, and the second physician is a treating physician or primary care provider.

20. The system of claim 18, wherein the at least one processor is further configured to execute the computer-readable instructions to interpret the input entered by the first physician and convert the input to standard nomenclature.

21. The system of claim 18, wherein the at least one processor is further configured to execute the computer-readable instructions to recognize informal nomenclature of a test, convert the informal nomenclature of the test to standardized nomenclature as used in a medical community, and ensure a correct test is ordered.

22. The system of claim 21, wherein the at least one processor is further configured to execute the computer-readable instructions to convert informal test names used by the facility to the Current Procedural Terminology (CPTs), each of the CPTs includes a five-digit number.

23. The system of claim 18, wherein the at least one processor is further configured to execute the computer-readable instructions to organize outstanding recommendations for the first physician in order to focus on next steps in patient care and treatment.

24. The system of claim 23 wherein the next steps correspond to at least one of calling the patient, calling either the first or second physician, transferring responsibility for care to a backup physician, ordering a recommended test, and receiving a new test result.
